(19) United States
(12) Patent Application Publication
(10) Pub. No.: US 2002/0123127 A1
(43) Pub. Date: Sep. 5, 2002

(54) Methods and compositions for reducing odor

(76) Inventor: Ling Y. Cheung, New Territories (CN)

Correspondence Address: FISH & NEAVE, 1251 AVENUE OF THE AMERICAS, 50TH FLOOR, NEW YORK, NY, 10020-1105, US

(21) Appl. No.: 09/797,372

(22) Filed: Mar. 1, 2001

(51) Int. Cl. C12N 1/14; C12N 1/16; C12N 1/18; C12N 15/01

(52) U.S. Cl. 435/254.21; 435/446

(57) ABSTRACT

Compositions comprising a plurality of yeast cells, wherein said plurality of yeast cells have been cultured in the presence of an alternating electric field having a specific frequency and a specific field strength for a period of time sufficient to substantially increase the capability of said plurality of yeast cells to reduce odor of a culture medium. Also included are methods of making such compositions.

### FIELD OF THE INVENTION

The invention relates to the use of yeast cells to reduce odor. These yeasts are useful in waste treatment, and can be obtained by growth in electromagnetic fields with specific frequencies and field strengths.

### BACKGROUND OF THE INVENTION

Environmental pollution by urban sewage and industrial waste water has posed a serious health threat to living organisms in the world. Currently, the most common methods for large-scale waste treatment, such as water treatment, include the activated sludge technology and the biomembrane technology. These technologies rely on the innate abilities of myriad natural microorganisms, such as fungi, bacteria and protozoa, to degrade pollutants. However, the compositions of these natural microbial components are difficult to control, affecting the reproducibility and quality of water treatment. Moreover, pathogenic microbes existing in these activated sludge or biomembranes cannot be selectively inhibited, and such microbes usually enter the environment with the treated water, causing “secondary pollution.”

Further, most of the current technologies cannot degrade harmful chemicals such as pesticides, insecticides, and chemical fertilizers. These technologies also cannot alleviate eutrophication, another serious environmental problem around the world. Eutrophication is usually caused by sewage, industrial waste water, fertilizers and the like. It refers to waters (e.g., a lake or pond) rich in minerals and organic nutrients that promote a proliferation of plant life, especially algae, which reduces the dissolved oxygen content or otherwise deteriorates water quality. Eutrophication often results in the extinction of other organisms.

### SUMMARY OF THE INVENTION

This invention is based on the discovery that certain yeast cells can be activated by electromagnetic fields of specific frequencies and field strengths to reduce odor of certain environmental pollutants. Compositions comprising these activated yeast cells can therefore be used for waste treatment, for example, treatment of sewage, industrial waste water, surface water, drinking water, sediment, soil, garbage, and manure, to deodorize the waste. Waste treatment methods using these compositions are more effective, efficient, and economical than the conventional methods.

This invention embraces a composition comprising a plurality of yeast cells that have been cultured in an alternating electric field having a frequency in the range of about 2160 to 2380 MHz (e.g., 2160-2250 or 2280-2380 MHz) and a field strength in the range of about 0.5 to 320 mV/cm (e.g., 40-260, 70-260, 80-250, 90-260, or 140-300 mV/cm). The yeast cells are cultured for a period of time sufficient to substantially increase the capability of said plurality of yeast cells to reduce odor in malodorous materials. In one embodiment, the frequency and/or the field strength of the alternating electric field can be altered within the aforementioned ranges during said period of time. In other words, the yeast cells can be exposed to a series of electromagnetic fields. An exemplary period of time is about 12 to 350 hours (e.g., 70-220, 70-320, 80-310, 85-220, 110-230, or 120-300 hours).

Yeast cells that can be included in this composition are available from the China General Microbiological Culture Collection Center (“CGMCC”), a depository recognized under the Budapest Treaty (China Committee for Culture Collection of Microorganisms, Institute of Microbiology, Chinese Academy of Sciences, Haidian, P.O. Box 2714, Beijing, 100080, China). Useful yeast species include, but are not limited to, Saccharomyces cerevisiae and Saccharomyces carlsbergensis. For instance, the yeast cells can be of the strain Saccharomyces cerevisiae Hansen AS2.53, AS2.163, AS2.396, AS2.397, AS2.423, AS2.452, AS2.502, AS2.516, AS2.541, AS2.558, AS2.559, AS2.560, AS2.561, AS2.562, AS2.607, AS2.612, IFFI 1052, IFFI 1202, IFFI 1213, IFFI 1247, or IFFI 1397; or Saccharomyces carlsbergensis Hansen AS2.605.

This invention also embraces a composition comprising a plurality of yeast cells, wherein said plurality of yeast cells have been activated such that they have a substantially increased capability to reduce odor of a culture medium as compared to unactivated yeast cells. Included in this invention are also methods of making these compositions.

As used herein, “reducing odor” or “deodorizing” refers to a process which results in a lower concentration of one or more odorous compounds. Odorous compounds include, but are not limited to, hydrogen sulfide, ammonium sulfide, other sulfur-containing compounds, ammonia, indole, methylindoles, pcresol, amines such as methylamine, dimethylamine and trimethylamine, and odorous organic acids, such as carboxylic acids, e.g., formic acid, acetic acid, propanoic acid and butyric acid, and other volatile fatty acids.

A “substantial increase” means an increase of more than 10 (e.g., 10², 10³, 10⁴, 10⁵, or 10⁶) fold.

A “culture medium” refers to a medium used in a laboratory for selecting and growing a given yeast strain, or to liquid or solid waste in need of treatment.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram showing an exemplary apparatus for activating yeast cells using electromagnetic fields. 1: yeast culture; 2: container; 3: power supply.

### DETAILED DESCRIPTION OF THE INVENTION

This invention is based on the discovery that certain yeast strains can be activated by electromagnetic fields (“EMF”) having specific frequencies and field strengths to become highly efficient in reducing foul odor of malodorous materials. Yeast cells having this function are defined herein as belonging to the same “functional group.” Compositions containing the activated yeast cells are useful in waste treatment.

Without being bound by any theory or mechanism, the inventor believes that EMFs activate or enhance the expression of a gene or a set of genes in yeast cells such that the yeast cells become active or more efficient in performing certain metabolic activities which lead to the desired odor-reducing result. The activated yeast cells may reduce odor by modifying or decomposing compounds that are malodorous.

I. Yeast Strains Useful in the Invention

The types of yeasts useful in this invention include, but are not limited to, yeasts of the genera of Saccharomyces, Schizosaccharomyces, Sporobolomyces, Torulopsis, Trichosporon, Wickerhamia, Ashbya, Blastomyces, Candida, Citeromyces, Crebrothecium, Cryptococcus, Debaryomyces, Endomycopsis, Eremothecium, Geotrichum, Hansenula, Kloeckera, Lipomyces, Pichia, Rhodosporidium, and Rhodotorula.

Exemplary species within the above-listed genera include, but are not limited to, Saccharomyces cerevisiae, Saccharomyces bailii, Saccharomyces carisbergensis, Saccharomyces chevalieri, Saccharomyces delbrueckii, Saccharomyces exiguus, Saccharomycesfermentati, Saccharomyces logos, Saccharomyces mellis, Saccharomyces microellipsoides, Saccharomyces oviformis, Saccharomyces rosei, Saccharomyces rouxii, Saccharomyces sake, Saccharomyces uvarum, Saccharomyces willianus, Saccharomyces sp., Saccharomyces ludwigii, Saccharomyces sinenses, Saccharomyces hailii, Saccharomyces carlsbergensis, Schizosaccharomyces octosporus, Schizosaccharomyces pombe, Sporobolomyces roseus, Sporobolomyces salmonicolor, Torulopsis candida, Torulopsis famta, Torulopsis globosa, Torulopsis inconspicua, Trichosporon behrendoo, Trichosporon capitatum, Trichosporon cutaneum, Wickerhamia fluoresens, Ashbya gossypii, Blastomyces dermatitidis, Candida albicans, Candida arborea, Candida guilliermondii, Candida krusei, Candida lambica, Candida lipolytica, Candida parakrusei, Candida parapsilosis, Candida pseudotropicalis, Candida pulcherrima, Candida robusta, Candida rugousa, Candida tropicalis, Candida utilis, Citeromyces matritensis, Crebrothecium ashbyii, Cryptococcus laurentii, Cryptococcus neoformans, Debaryomyces hansenii, Debaryomyces kloeckeri, Debaryomyces sp., Endomycopsisfibuligera, Eremothecium ashbyii, Geotrichum candidum, Geotrichum ludwigii, Geotrichum robustum, Geotrichum suaveolens, Hansenula anomala, Hansenula arabitolgens, Hansenula jadinii, Hansenula saturnus, Hansenula schneggii, Hansenula subpelliculosa, Kloeckera apiculata, Lipomyces starkeyi, Pichiafarinosa, Pichia membranaefaciens, Rhodosporidium toruloides, Rhodotorula aurantiaca, Rhodotorula glutinis, Rhodotorula minuta, Rhodotorula rubar, and Rhodotorula sinesis.

Yeast strains useful for this invention can be obtained from laboratory cultures, or from publically accessible culture depositories, such as CGMCC and the American Type Culture Collection, 10801 University Boulevard, Manassas, Va. 20110-2209. Non-limiting examples of useful strains (with accession numbers of CGMCC) are Saccharomyces cerevisiae Hansen AS2.53, AS2.163, AS2.396, AS2.397, AS2.423, AS2.452, AS2.502, AS2.516, AS2.541, AS2.558, AS2.559, AS2.560, AS2.561, AS2.562, AS2.607, AS2.612, IFFI 1052, IFFI 1202, IFFI 1213, IFFI 1247, and IFFI 1397; and Saccharomyces carlsbergensis Hansen AS2.605.

Although it is preferred, the preparation of the yeast compositions of this invention is not limited to starting with a pure strain of yeast. A yeast composition of the invention may be produced by culturing a mixture of yeast cells of different species or strains that have the same odor-reducing function. The ability of any species or strain of yeasts to perform this function can be readily tested by methods known in the art.

Certain yeast species that can be activated according to the present invention are known to be pathogenic to human and/or other living organisms. These yeast species include, for example, Ashbya gossypii, Blastomyces dermatitidis, Candida albicans, Candida parakrusei, Candida tropicalis, Citeromyces matritensis, Crebrothecium ashbyii, Cryptococcus laurentii, Cryptococcus neoformans, Debaryomyces hansenii, Debaryomyces kloeckeri, Debaryomyces sp., and Endomycopsis fibuligera. Under certain circumstances, it may be less preferable to use such pathogenic yeasts in this invention. If use of these species is necessary, caution should be exercised to minimize the leak of the yeast cells into the final treatment product that enters the environment.

II. Application of Electromagnetic Fields

An electromagnetic field useful in this invention can be generated and applied by various means well known in the art. For instance, the EMF can be generated by applying an alternating electric field or an oscillating magnetic field.

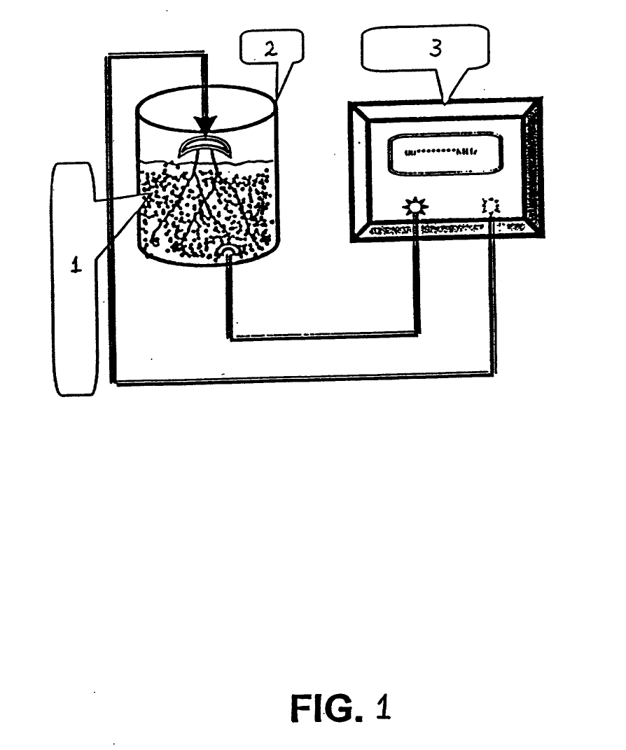

Alternating electric fields can be applied to cell cultures through electrodes in direct contact with the culture medium, or through electromagnetic induction. See, e.g., FIG. 1. Relatively high electric fields in the medium can be generated using a method in which the electrodes are in contact with the medium. Care must be taken to prevent electrolysis at the electrodes from introducing undesired ions into the culture and to prevent contact resistance, bubbles, or other features of electrolysis from dropping the field level below that intended. Electrodes should be matched to their environment, for example, using Ag-AgCl electrodes in solutions rich in chloride ions, and run at as low a voltage as possible. For general review, see Goodman et al., Effects of EMF on Molecules and Cells, International Review of Cytology, A Survey of Cell Biology, Vol. 158, Academic Press, 1995.

The EMFs useful in this invention can also be generated by applying an oscillating magnetic field. An oscillating magnetic field can be generated by oscillating electric currents going through Helmholtz coils. Such a magnetic field in turn induces an electric field.

The frequencies of EMFs useful in this invention range from 5 to 5000 MHz, e.g., from 2160 MHz to 2380 MHz (e.g., 2160-2250 MHz or 2280-2380 MHz). Exemplary frequencies are 2160, 2165, 2170, 2175, 2180, 2185, 2190, 2195, 2200, 2205, 2210, 2215, 2220, 2225, 2230, 2235, 2240, 2245, 2250, 2280, 2285, 2290, 2295, 2300, 2305, 2310, 2315, 2320, 2325, 2330, 2335, 2340, 2345, 2350, 2355, 2360, 2365, 2370, 2375, and 2380 MHz.

The field strength of the electric field useful in this invention ranges from about 0.5 to 320 mV/cm, e.g. from 30 to 310 mV/cm (e.g., 40-260, 70-260, 80-250, 90-260, or 140-300 mV/cm). Exemplary field strengths are 98, 240, 250, and 290 mV/cm.

When a series of EMFs are applied to a yeast culture, the yeast culture can remain in the same container while the same set of EMF generator and emitters is used to change the frequency and/or field strength. The EMFs in the series can each have a different frequency or a different field strength; or a different frequency and a different field strength. Such frequencies and field strengths are preferably within the above-described ranges. In one embodiment, an EMF at the beginning of the series has a field strength identical to or lower than that of a subsequent EMF, such that the yeast cell culture is exposed to EMFs of progressively increasing field strength. Although any practical number of EMFs can be used in a series, it may be preferred that the yeast culture be exposed to a total of 2, 3, 4, 5, 6, 7, 8, 9 or 10 EMs in a series.

By way of example, the yeast cells can be cultured in a series of alternating electric fields each having a frequency in the range of 2160 to 2250 MHz or 2280 to 2380 MHz and a field strength in the range of 30 to 310 mV/cm. The yeast cells are exposed to each EMF for about 10 to 40 hours. Preferably, the field strength remains the same in the series whereas the frequency progressively increases.

Alternatively, the yeast cells can be cultured in a first series of alternating electric fields each having a frequency in the range of 2280 to 2380 MHz and a field strength in the range of 90 to 260 mV/cm. The yeast cells are exposed to each EMF for about 15 to 20 hours. After culturing in the first series of EMFs, the resultant yeast cells are further incubated in a second series of alternating electric fields for a total of 20 to 50 hours. It may be preferred that the frequencies in the second series of alternating electric fields are identical to those of the first series in sequence and the field strengths in the second series are increased to a higher level within the range of 90 to 260 mV/cm.

Although the yeast cells can be activated after even a few hours of culturing in the presence of an EMF, it may be preferred that the activated yeast cells be allowed to multiply and grow in the presence of the EMF(s) for a total of 70-220, 70-320, 80-310, 85-220, 110-230, or 120-300 hours.

FIG. 1 illustrates an exemplary apparatus for generating alternating electric fields. An electric field of a desired frequency and intensity is generated by an AC source (3) capable of generating an alternating electric field, preferably in a sinusoidal wave form, in the frequency range of 5 to 5000 MHz. Signal generators capable of generating signals with a narrower frequency range can also be used. If desirable, a signal amplifier can also be used to increase the output. The alternating electric field can be applied to the culture by a variety of means including placing the yeast culture in close proximity to the signal emitters. In one embodiment, the electric field is applied by electrodes submerged in the culture (1). In this embodiment, one of the electrodes can be a metal plate placed on the bottom of the container (2), and the other electrode can comprise a plurality of electrode wires evenly distributed in the culture (1) so as to achieve even distribution of the electric field energy. The number of electrode wires used depends on the volume of the culture as well as the diameter of the wires. In a preferred embodiment, for a culture having a volume up to 5000 ml, one electrode wire having a diameter of 0.1 to 1.2 mm can be used for each 100 ml of culture. For a culture having a volume greater than 1000 L, one electrode wire having a diameter of 3 to 30 mm can be used for each 1000 L of culture.

III. Culture Media

Culture media useful in this invention contain sources of nutrients assimilable by yeast cells. In this invention, a culture medium refers to a laboratory culture medium, or liquid or solid waste in need of treatment. Complex carbon-containing substances in a suitable form, such as carbohydrates (e.g., sucrose, glucose, fructose, dextrose, maltose, xylose, cellulose, starches, etc.) and coal, can be the carbon sources for yeast cells. The exact quantity of the carbon sources utilized in the medium can be adjusted in accordance with the other ingredients of the medium. In general, the amount of carbohydrates varies between about 0.1% and 5% by weight of the medium and preferably between about 0.1% and 2%, and most preferably about 1%. These carbon sources can be used individually or in combination. Among the inorganic salts which can be added to the culture medium are the customary salts capable of yielding sodium, potassium, calcium, phosphate, sulfate, carbonate, and like ions. Non-limiting examples of nutrient inorganic salts are (NH₄)₂HPO₄, KH₂PO₄, K₂HPO₄, CaCO₃, MgSO₄, NaCl, and CaSO₄.

IV. Electromagnetic Activation of Yeast Cells

Yeasts of this invention reduce odor by lowering the concentration of malodorous materials. Malodorous materials include, but are not limited to, hydrogen sulfide, ammonium sulfide, other sulfur-containing compounds, ammonia, indole, methylindoles, p-cresol, amines such as methylamine, dimethylamine and trimethylamine, and odorous organic acids, such as carboxylic acids, e.g., formic acid, acetic acid, propanoic acid and butyric acid, and other volatile fatty acids.

To activate the innate ability of yeast cells to reduce odor, these cells can be cultured in an appropriate medium under sterile conditions at 25° C.-30° C., e.g., 28° C., for a sufficient amount of time, e.g., 12-350 hours (for example, 70-220, 70-320, 80-310, 85-220, 110-230, or 120-300 hours), in an alternating electric field or a series of alternating electric fields as described above. The culturing process may preferably be conducted under conditions in which the concentration of dissolved oxygen is between 0.025 to 0.8 mol/m³, preferably 0.4 mol/m³. The oxygen level can be controlled by, for example, stirring and/or bubbling.

An exemplary culture medium contains in per 1000 ml of sewage water (containing malodorous materials): 0.2 g of NaCl, 0.2 g of MgSO₄·7H₂O, 0.5 g of CaCO₃·5H₂O, 0.2 g of CaSO₄·2H₂O, and 0.5 g of K₂HPO₄.

Subsequently, the yeast cells can be measured for their ability to reduce odor. Various methods and techniques are known to measure the intensity of an odor, including but not limited to gas chromatography, HPLC, and mass spectrometry. A reduction of the intensity of the odor of malodorous materials can also be determined subjectively. One subjective measurement of odor intensity is to measure the dilution necessary so that the odor is imperceptible or doubtful to a human or animal test panel. Any methods and techniques for objectively or subjectively determining the intensity of an odor can be used to monitor the ability of the yeast compositions to reduce odor.

In an exemplary method, sewage water containing about 2 g/L methylamine/dimethylamine/trimethylamine, 1 g/L indole, 2 g/L p-cresol, 1 g/L hydrogen sulfide, 2 g/L acetic acid and/or 1 g/L ammonia is used as a substrate. The sewage is inoculated with a dry yeast cell preparation, at a concentration of 0.2-0.6 g/L, and cultured for 24 hours at 10-35° C. The level of the malodorous chemical is measured by gas chromatography. The difference between the levels of the above-mentioned malodorous components before and after 24 hours indicates the odor-reducing ability of the yeast cells.

Essentially the same protocol as described above can be used to grow activated yeast cells. To initiate the process, each 100 ml of culture medium is inoculated with yeast cells of the same functional group at a density of 10²-10⁵ cells/ml, preferably 3×10²-10⁴ cells/ml. The culturing process is carried out at 20-40° C., preferably at about 25-28° C., for 48-96 hours. The process can be scaled up or down according to needs. For an industrial scale of production, seventy-five liters of a sterile culture medium are inoculated with the yeast cells. This culture medium consists of 10 L of the culture medium described above for this particular yeast functional group, 30 kg of starch, and 65 L of distilled water. At the end of the culturing process, the yeast cells may preferably reach a concentration of 2×10¹⁰ cells/ml. The cells are recovered from the culture by various methods known in the art, and stored at about 15-20° C. The yeast should be dried within 24 hours and stored in powder form.

V. Acclimatization of Yeast Cells to Waste Environment

In yet another embodiment of the invention, the yeast cells may also be cultured under certain conditions so as to acclimatize the cells to a particular type of waste. This acclimatization process results in better growth and survival of the yeasts in a particular waste environment.

To achieve this, the yeast cells of a given functional group can be mixed with waste material from a particular source at 10⁶ to 10⁸ cells (e.g., 10⁷ cells) per 1000 ml. The yeast cells are then exposed to an alternating electric field as described above. The strength of the electric field can be 100 to 400 mV/cm (e.g., 120 to 250 mV/cm). The culture is incubated at temperatures that cycle between about 5° C. to about 45° C. at a 5° C. increment. For example, in a typical cycle, the temperature of the culture may start at 5° C. and be kept at this temperature for about 1-2 hours, then adjusted up to 10° C. and kept at this temperature for 1-2 hours, then adjusted to 15° C. and kept at this temperature for about 1-2 hours, and so on and so forth, until the temperature reaches 45° C. Then the temperature is brought down to 40° C. and kept at this temperature for about 1-2 hours, and then to 35° C. and kept at this temperature for about 1-2 hours, and so on and so forth, until the temperature returns to 5° C. The cycles are repeated for about 48-96 hours. The resulting yeast cells are then dried and stored at 0-4° C.

VI. Manufacture of the Waste Treatment Compositions

The yeast cells of this invention can be mixed with an appropriate filler, such as rock powder and coal ash at the following ratio: 600 L of yeast cell culture at 2×10¹⁰ cells/ml and 760 kg of filler materials. The mixture is quickly dried at a temperature below 65° C. for 10 minutes in a dryer, and then further dried at a temperature below 70° C. for no more than 30 minutes so that the water content is less than 7%. The dried composition is then cooled to room temperature for packaging.

These dried yeast compositions may be used to treat polluted surface water, sewage, or any other type of waste water. To treat polluted surface water, a yeast solution may be prepared by adding 1 kg of the dried yeast composition to 30 L of clean water. The yeast solution is then sprayed onto the polluted surface water at about 1-3 L of the solution per square meter of the polluted surface water. To treat sewage or any other type of waste water, a yeast solution may be prepared by adding about 1 kg of the dried yeast composition to 10-30 L of clean water. The yeast solution is incubated at 10-35° C. for 24-48 hours. The resultant yeast solution is then added to the waste water at about 3-20 L of the solution per liter of waste water.

### VII. EXAMPLES

In order that this invention be more fully understood, the following examples are set forth. These examples are for the purpose of illustration only and are not to be construed as limiting the scope of the invention in any way.

### Example 1

### Reduction of Odor Caused by Hydrogen Sulfide

Saccharomyces cerevisiae Hansen Var. ellipsoideus (Hansen) Dekker AS2.559 cells were cultured in the presence of a series of alternating electric fields in the following sequence: the yeast cells were exposed to (1) an alternating electric field having a frequency of 2165 MHz and a field strength of 240 mV/cm for 20 hours; (2) then to an alternating electric field having a frequency of 2175 MHz and a field strength of 240 mV/cm for 20 hours; (3) then to an alternating electric field having a frequency of 2200 MHz and a field strength of 240 mV/cm for 20 hours; and (4) finally to an alternating electric field having a frequency of 2235 MHz and a field strength of 240 mV/cm for 20 hours.

To test the ability of the EMF-treated AS2.559 cells to reduce odor caused by hydrogen sulfide, waste water or filtrate from animal manure or garbage was supplemented with H₂S to reconstitute a solution containing H₂S at 100 mg/L. 0.1 ml of the EMF-treated AS2.559 cells at a concentration higher than 10⁸ cells/ml was added to 100 L of the H₂S solution and cultured at 28° C. for 24 hours (solution A). One hundred liters of the H₂S solution containing the same number of non-treated yeast cells (solution B) or containing no yeast cells (solution C) were used as controls. After 24 hours of incubation, the solutions were examined using mass spectrometry (MAS-nose, manufactured by VG). The results showed that after 24 hours of incubation, the H₂S concentration of solution A decreased more than 13% relative to solution C. In contrast, the H₂S concentration of solution B had no significant change relative to solution C.

### Example 2

### Reduction of Odor Caused by Ammonia

Saccharomyces cerevisiae Hansen AS2.423 cells were cultured in the presence of a series of alternating electric fields in the following sequence: the yeast cells were exposed to (1) an alternating electric field having a frequency of 2160 MHz and a field strength of 250 mV/cm for 20 hours; (2) then to an alternating electric field having a frequency of 2175 MHz and a field strength of 250 mV/cm for 20 hours; (3) then to an alternating electric field having a frequency of 2210 MHz and a field strength of 250 mV/cm for 20 hours; and (4) finally to an alternating electric field having a frequency of 2245 MHz and a field strength of 250 mV/cm for 10 hours.

To test the ability of the EMF-treated AS2.423 cells to reduce odor caused by ammonia, waste water or filtrate from animal manure or garbage was supplemented with ammonia to reconstitute a solution containing ammonia (in the form of ammonium hydroxide) at 100 mg/L. 0.1 ml of the EMF-treated AS2.423 cells at a concentration higher than 10⁸ cells/ml was added to 100 L of the ammonia solution and cultured at 28° C. for 24 hours (solution A). One hundred liters of the ammonia solution containing the same number of non-treated yeast cells (solution B) or containing no yeast cells (solution C) were used as controls. After 24 hours of incubation, the solutions were examined using mass spectrometry (MAS-nose, manufactured by VG). The results showed that after 24 hours of incubation, the ammonia concentration of solution A decreased more than 11% relative to solution C. In contrast, the ammonia concentration of solution B had no significant change relative to solution C.

### Example 3

### Reduction of Odor Caused by Indole

Saccharomyces cerevisiae Hansen Var. ellipsoideus (Hansen) Dekker AS2.612 cells were cultured in the presence of a series of alternating electric fields in the following sequence: the yeast cells were exposed to (1) an alternating electric field having a frequency of 2165 MHz and a field strength of 240 mV/cm for 40 hours; (2) then to an alternating electric field having a frequency of 2180 MHz and a field strength of 240 mV/cm for 20 hours; (3) then to an alternating electric field having a frequency of 2200 MHz and a field strength of 240 mV/cm for 40 hours; and (4) finally to an alternating electric field having a frequency of 2220 MHz and a field strength of 240 mV/cm for 20 hours.

To test the ability of the EMF-treated AS2.612 cells to reduce odor caused by indole, waste water or filtrate from animal manure or garbage was supplemented with indole to reconstitute a solution containing indole at 100 mg/L. 0.1 ml of the EMF-treated AS2.612 cells at a concentration higher than 10⁸ cells/ml was added to 100 L of the indole solution and cultured at 28° C. for 24 hours (solution A). One hundred liters of the indole solution containing the same number of non-treated yeast cells (solution B) or containing no yeast cells (solution C) were used as controls. After 24 hours of incubation, the solutions were examined using mass spectrometry (MAS-nose, manufactured by VG). The results showed that after 24 hours of incubation, the indole concentration of solution A decreased more than 15% relative to solution C. In contrast, the indole concentration of solution B had no significant change relative to solution C.

### Example 4

### Reduction of Odor Caused by Methylamine, Dimethylamine or Trimethylamine

Saccharomyces cerevisiae Hansen Var. ellipsoideus (Hansen) Dekker AS2.541 cells were cultured in the presence of a series of alternating electric fields in the following sequence: the yeast cells were exposed to (1) an alternating electric field having a frequency of 2160 MHz and a field strength of 250 mV/cm for 20 hours; (2) then to an alternating electric field having a frequency of 2190 MHz and a field strength of 250 mV/cm for 10 hours; (3) then to an alternating electric field having a frequency of 2210 MHz and a field strength of 250 mV/cm for 40 hours; and (4) finally to an alternating electric field having a frequency of 2250 MHz and a field strength of 250 mV/cm for 40 hours.

To test the ability of the EMF-treated AS2.541 cells to reduce odor caused by methylamine, dimethylamine or trimethylamine, waste water or filtrate from animal manure or garbage was supplemented with methylamine, dimethylamine, or trimethylamine to reconstitute a solution containing methylamine, dimethylamine, or trimethylamine at 100 mg/L. 0.1 ml of the EMF-treated AS2.541 cells at a concentration higher than 10⁸ cells/ml was added to 100 L of the methylamine, dimethylamine or trimethylamine solution and cultured at 28° C. for 24 hours (solution A). One hundred liters of the methylamine, dimethylamine or trimethylamine solution containing the same number of non-treated yeast cells (solution B) or containing no yeast cells (solution C) were used as controls. After 24 hours of incubation, the solutions were examined using mass spectrometry (MAS-nose, manufactured by VG). The results showed that after 24 hours of incubation, the methylamine, dimethylamine or trimethylamine concentration of solution A decreased more than 23% relative to solution C. In contrast, the methylamine, dimethylamine, or trimethylamine concentration of solution B had no significant change relative to solution C.

### Example 5

### Reduction of Odor Caused by Organic Acids

Saccharomyces cerevisiae Hansen Var. ellipsoideus (Hansen) Dekker AS2.53 cells were cultured in the presence of a series of alternating electric fields in the following sequence: the yeast cells were exposed to (1) an alternating electric field having a frequency of 2315 MHz and a field strength of 290 mV/cm for 30 hours; (2) then to an alternating electric field having a frequency of 2335 MHz and a field strength of 290 mV/cm for 10 hours; (3) then to an alternating electric field having a frequency of 2355 MHz and a field strength of 290 mV/cm for 20 hours; and (4) finally to an alternating electric field having a frequency of 2375 MHz and a field strength of 290 mV/cm for 10 hours.

To test the ability of the EMF-treated AS2.53 cells to reduce odor caused by organic acids, waste water or filtrate from animal manure or garbage was supplemented with acetic acid to reconstitute a solution containing acetic acid at 100 mg/L. 0.1 ml of the EMF-treated AS2.53 cells at a concentration higher than 10⁸ cells/ml was added to 100 L of the acetic acid solution and cultured at 28° C. for 24 hours (solution A). One hundred liters of the acetic acid solution containing the same number of non-treated yeast cells (solution B) or containing no yeast cells (solution C) were used as controls. After 24 hours of incubation, the solutions were examined using mass spectrometry (MAS-nose, manufactured by VG). The results showed that after 24 hours of incubation, the acetic acid concentration of solution A decreased more than 19% relative to solution C. In contrast, the acetic acid concentration of solution B had no significant change relative to solution C.

### Example 6

### Reduction of Odor Caused by p-Cresol

Saccharomyces cerevisiae Hansen Var. ellipsoideus (Hansen) Dekker AS2.163 cells were cultured in the presence of a series of alternating electric fields in the following sequence: the yeast cells were exposed to (1) an alternating electric field having a frequency of 2300 MHz and a field strength of 98 mV/cm for 20 hours; (2) then to an alternating electric field having a frequency of 2370 MHz and a field strength of 98 mV/cm for 15 hours; (3) then to an alternating electric field having a frequency of 2300 MHz and a field strength of 250 mV/cm for 20 hours; and (4) finally to an alternating electric field having a frequency of 2370 MHz and a field strength of 250 mV/cm for 30 hours.

To test the ability of the EMF-treated AS2.163 cells to reduce odor caused by p-cresol, waste water or filtrate from animal manure or garbage was supplemented with p-cresol to reconstitute a solution containing p-cresol at 100 mg/L. 0.1 ml of the EMF-treated AS2.163 cells at a concentration higher than 10⁸ cells/ml was added to 100 L of the p-cresol solution and cultured at 28° C. for 24 hours (solution A). One hundred liters of the p-cresol solution containing the same number of non-treated yeast cells (solution B) or containing no yeast cells (solution C) were used as controls. After 24 hours of incubation, the solutions were examined using mass spectrometry (MAS-nose, manufactured by VG). The results showed that after 24 hours of incubation, the p-cresol concentration of solution A decreased more than 23% relative to solution C. In contrast, the p-cresol concentration of solution B had no significant change relative to solution C.

While a number of embodiments of this invention have been set forth, it is apparent that the basic constructions may be altered to provide other embodiments which utilize the compositions and methods of this invention.

What is claimed is:

1. A composition comprising a plurality of yeast cells, wherein said plurality of yeast cells have been cultured in the presence of an alternating electric field having a frequency in the range of 2160 to 2380 MHz and a field strength in the range of 0.5 to 320 mV/cm for a period of time sufficient to substantially increase the capability of said plurality of yeast cells to reduce odor of a culture medium.

2. The composition of claim 1, wherein said frequency is in the range of 2160 to 2250 MHz or 2280 to 2380 MHz.

3. The composition of claim 1, wherein said yeast cells are cells of the species Saccharomyces cerevisiae or Saccharomyces carlsbergensis.

4. The composition of claim 1, wherein said yeast cells are cells of the strain deposited at The China General Microbiological Culture Collection Center with an accession number selected from the group consisting of AS2.53, AS2.163, AS2.396, AS2.397, AS2.423, AS2.452, AS2.502, AS2.516, AS2.541, AS2.558, AS2.559, AS2.560, AS2.561, AS2.562, AS2.605, AS2.607, AS2.612, IFFI 1052, IFFI 1202, IFFI 1213, IFFI 1247, and IFFI 1397.

5. The composition of claim 1, wherein said odor is caused by hydrogen sulfide.

6. The composition of claim 5, wherein said frequency is in the range of 2160 to 2250 MHz, and said field strength is in the range of 80 to 250 mV/cm.

7. The composition of claim 1, wherein said odor is caused by ammonia.

8. The composition of claim 7, wherein said frequency is in the range of 2160 to 2250 MHz, and said field strength is in the range of 70 to 260 mV/cm.

9. The composition of claim 1, wherein said odor is caused by indole.

10. The composition of claim 9, wherein said frequency is in the range of 2160 to 2250 MHz, and said field strength is in the range of 70 to 260 mV/cm.

11. The composition of claim 1, wherein said odor is caused by methylamine, dimethylamine, or trimethylamine.

12. The composition of claim 11, wherein said frequency is in the range of 2160 to 2250 MHz, and said field strength is in the range of 40 to 260 mV/cm.

13. The composition of claim 1, wherein said odor is caused by organic acids.

14. The composition of claim 13, wherein said frequency is in the range of 2280 to 2380 MHz, and said field strength is in the range of 140 to 300 mV/cm.

15. The composition of claim 14, wherein said odor is caused by acetic acid.

16. The composition of claim 1, wherein said odor is caused by p-cresol.

17. The composition of claim 16, wherein said frequency is in the range of 2280 to 2380 MHz, and said field strength is in the range of 90 to 260 mV/cm.

18. A composition comprising a plurality of yeast cells, wherein said plurality of yeast cells have been activated such that they have a substantially increased capability to reduce odor of a culture medium as compared to unactivated yeast cells.

19. A method of preparing a yeast composition, comprising culturing a plurality of yeast cells in the presence of an alternating electric field or multiple alternating electric fields having a frequency in the range of 2160 to 2380 MHz and a field strength in the range of 0.5 to 320 mV/cm for a period of time sufficient to substantially increase the capability of said plurality of yeast cells to reduce odor of a culture medium.
